(19) United States
(12) Patent Application Publication
(10) Pub. No.: US 2025/0090379 A1
(43) Pub. Date: Mar. 20, 2025

(54) SINGLE-PIECE PATIENT INTERFACE DEVICE FOR OPHTHALMIC LASER SURGERY

(71) Applicant: AMO Development, LLC, Irvine, CA (US)

(72) Inventors: Reena Pathania, Fremont, CA (US); Aaron Quinn, San Leandro, CA (US); Hong Fu, Pleasanton, CA (US); Daryl Wong, San Jose, CA (US)

(21) Appl. No.: 18/888,079

(22) Filed: Sep. 17, 2024

Related U.S. Application Data
(60) Provisional application No. 63/583,455, filed on Sep. 18, 2023.

(51) Int. Cl. A61F 9/009; A61B 17/30

(52) U.S. Cl. CPC A61F 9/009; A61B 2017/306

(57) ABSTRACT

A single-piece patient interface device for coupling a patient's eye to an ophthalmic surgical laser system includes a cone substrate with a rigid frustoconical shaped shell for coupling to the laser system, and a flexible suction ring integrally joined to the lower end of the rigid shell for coupling to the patient's eye. The suction ring has a circular skirt extending downwardly from a base portion, a diaphragm extending from the base portion and disposed inside of the skirt, and a contact lens held by the diaphragm to cover a center opening. When the skirt contacts the eye's surface, the skirt, the diaphragm, the contact lens and the eye surface form a vacuum chamber, where a vacuum may be applied to secure the patient interface device to the eye. The parameters of the suction ring are optimized to fit a large range of eye sizes, including smaller eyes.

### CROSS-REFERENCE TO RELATED APPLICATIONS

This application claims priority to U.S. Provisional Application No. 63/583,455, filed Sep. 18, 2023, the entire contents of which are hereby incorporated by reference in its entirety.

### BACKGROUND OF THE INVENTION

This invention relates to ophthalmic surgical laser systems, and in particular, it relates to a patient interface device used to stabilize the patient's eye and to deliver the laser beam to the eye during ophthalmic surgery.

Laser-assisted eye surgeries are common procedures for vision correction, cataract treatment, etc. During such procedures, a series of laser pulses are precisely delivered into the cornea, lens, or other tissues of the eye to create incisions via photo-dissection. The patient's eye must be connected to the laser system prior to this type of surgery and maintained at a stationary position during the laser treatment to eliminate any chance of eye movements. Rolling of the eye or any shifts in eye position may lead to termination of the procedure by the surgeon or post-surgery complications such as delayed visual acuity recovery, vision aberrations, and coma.

In some current ophthalmic surgical laser systems, the laser instrument and the patient eye are connected through a two-piece patient interface device, which includes a lens cone holding a contact lens and a suction ring with a flexible skirt. During an eye docking procedure prior to starting the laser treatment, the suction ring is coupled to the eye by applying a vacuum to the suction ring, the lens cone is coupled to the laser delivery head of the laser system, and the two pieces are assembled and locked to each other. Some examples of two-piece patient interface devices are described in commonly owned U.S. patents application Ser. Nos. 17/663,198 filed on May 12, 2022 and 18/054,915 filed on Nov. 14, 2022. While two-piece patient interface devices have their advantages, the assembly of the two pieces during the docking procedure takes time and, in some situations, the two pieces are still able to undergo some extent of relative motions, resulting in eye movement relative to the laser system.

Some other ophthalmic surgical laser systems employ single-piece patient interface devices, where a single-piece structure is coupled to both the eye and the laser system. Some examples of single-piece patient interface devices are described in commonly owned U.S. Pat. Appl. Pub. No. 20170281407, entitled “Patient interface device for laser eye surgery having light guiding structure for illuminating eye,” No. 20180116870, entitled “Patient interface device for ophthalmic surgical laser system”, and No. 20180303664, entitled “Patient interface device for ophthalmic surgical laser system”. Single-piece patient interface devices can improve workflow, reduce procedure time, reduce the number of parts required for surgery, reduce the chance for eye movement, and improve ease of use.

Patient interface devices with scleral support features have been described, for example, in commonly owned U.S. Pat. Appl. Pub. No. 20230149217, entitled “Patient interface for ophthalmic laser surgery employing scleral support structures to reduce intraocular pressure”.

### SUMMARY OF THE INVENTION

Embodiments of the present invention provide a single-piece patient interface device that is optimized to fit a larger range of eye sizes, including smaller eyes, while reducing undesirable effects during docking.

Additional features and advantages of the invention will be set forth in the descriptions that follow and in part will be apparent from the description, or may be learned by practice of the invention. The objectives and other advantages of the invention will be realized and attained by the structure particularly pointed out in the written description and claims thereof as well as the appended drawings.

To achieve the above objects, the present invention provides a patient interface device for coupling a patient's eye to an ophthalmic surgical laser system, which includes: a cone substrate, including a rigid frustoconical shaped shell, configured to be coupled to the ophthalmic surgical laser system; and a flexible suction ring joined to a lower end of the rigid shell, wherein the cone substrate and the suction ring are formed integrally as a single piece, wherein the suction ring includes a base portion joined to the lower end of the rigid shell, a circular skirt extending downwardly from the base portion, a circular diaphragm extending from the base portion and disposed inside of the skirt, and a contact lens joined to the diaphragm and disposed in a center opening of the diaphragm, and wherein when the skirt contacts a surface of the patient's eye, the skirt, the diaphragm, the contact lens and the surface of the eye form a vacuum chamber.

In some embodiments, an outer diameter of the skirt is between 14 mm and 21 mm, a skirt length defined as a vertical distance from a lower surface of the contact lens to a distal end of the skirt is between 3 mm and 6.5 mm, a thickness of the skirt is between 0.25 mm and 3 mm, and a stiffness of the skirt is between 20A and 70A Shore durometer. In some embodiments, the outer diameter of the skirt is between 15 mm and 19 mm, the skirt length is between 5.47 mm and 5.52 mm, the thickness of the skirt is between 0.42 and 0.62 mm, and the stiffness of the skirt is between 30A and 42A Shore durometer. In one particular example, the outer diameter of the skirt is 19 mm, the skirt length is 5.5 mm, and the thickness of the skirt is 0.5 mm.

In some embodiments, the skirt is vertical; in some other embodiments, the skirt has a flare of 5-8 degree. In some embodiments, the suction ring further includes a plurality of scleral support pads joined to the diaphragm and extending downwardly and evenly spaced in a circumferential direction, wherein a bottom surface of each scleral support pads is sloped, being lower at a radially outer end and higher at a radially inner end.

It is to be understood that both the foregoing general description and the following detailed description are exemplary and explanatory and are intended to provide further explanation of the invention as claimed.

### BRIEF DESCRIPTION OF DRAWINGS

FIG. 1 is a top perspective view showing a single-piece patient interface device according to an embodiment of the present invention.

FIG. 2 is a bottom plan view of the patient interface device of FIG. 1.

FIG. 3 is a bottom perspective view of the suction ring of the patient interface device of FIG. 1.

FIG. 4 is a side cut-away and cross-section view of the suction ring of the patient interface device of FIG. 1.

FIG. 5 illustrates the patient interface device of FIG. 1 affixed to a patient's eye.

FIG. 6A is a cross-sectional view which schematically illustrates a part of the suction ring of a patient interface device according to one embodiment of the present invention.

FIG. 6B is a cross-sectional view which schematically illustrates the part of the suction ring of FIG. 6A holding a contact lens, affixed to the eye and applanating the cornea.

FIG. 7A is a cross-sectional view which schematically illustrates a part of the suction ring of a patient interface device according to another embodiment of the present invention.

FIG. 7B is a cross-sectional view which schematically illustrates the part of the suction ring of FIG. 7A holding a contact lens, affixed to the eye and applanating the cornea.

FIGS. 8A-8E schematically illustrate a portion of the patient interface device of FIG. 1, coupled to the laser delivery head, in various spatial relationships relative to various eye models. FIG. 8A shows the patient interface device with a model of a normal sized eye, without corneal applanation. FIG. 8B shows the patient interface device with the model of the normal sized eye, with some corneal applanation so that a seal is formed with the eye surface but before vacuum suction is applied. FIG. 8C shows the patient interface device with a model of a smaller sized eye, without corneal applanation. FIG. 8D shows the patient interface device with the model of the smaller sized eye, with some corneal applanation so that a seal is formed with the eye surface but before vacuum suction is applied. FIG. 8E shows the suction ring with a model of a larger sized eye, where a seal is formed with the eye surface without corneal applanation, and before vacuum suction is applied.

### DETAILED DESCRIPTION OF THE INVENTION

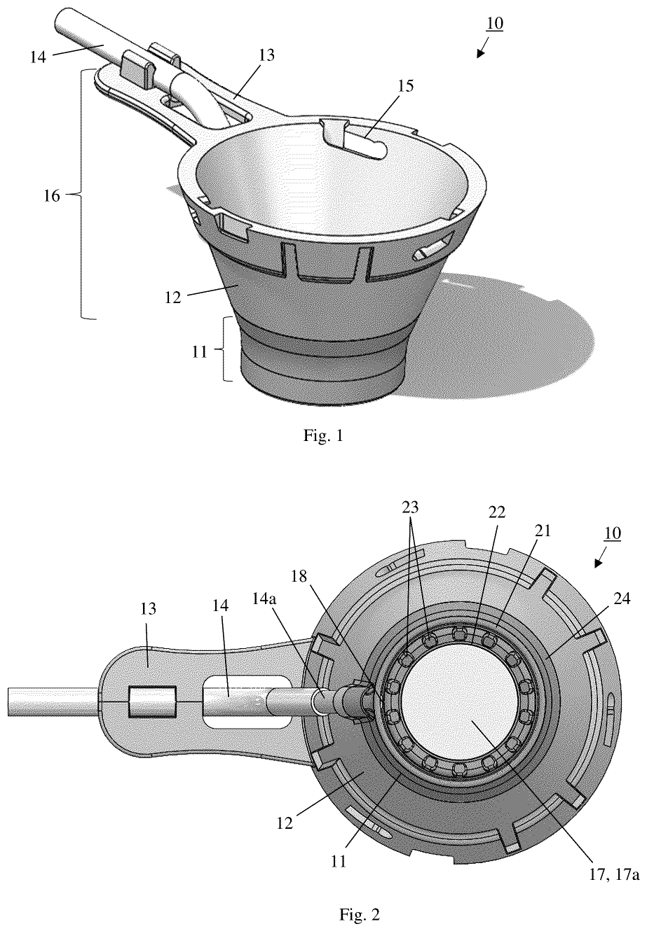

A single-piece patient interface device according to an embodiment of the present invention is described with reference to FIGS. 1-5. FIG. 1 is a perspective view, and FIG. 2 is a bottom plan view, of the patient interface device 10. The patient interface device 10 is a single-piece structure, which includes a cone substrate 16 and a suction ring 11 formed integrally as a single piece. The cone substrate 16 includes a rigid frustoconical shaped shell 12 defining a larger upper opening 12a and a smaller lower opening; the suction ring 11 is joined to the lower end of the rigid shell. The cone substrate 16 also includes a tubing guide arm 13 that extends from the shell 12. The cone substrate 16 is preferably formed of a hard plastic material such as polycarbonate or other suitable materials. The suction ring 11 is preferably formed of a soft, flexible material such as a soft thermoplastic elastomer (TPE) material, or other suitable flexible and compliant materials such as silicone, flexible plastic, rubber, etc. The suction ring 11 and the cone substrate 16 are integrally joined together, preferably by overmolding, which eliminates adhesives, but they may alternatively be joined together by adhesives or other means. The patient interface device is a single-piece device in that its parts are not intended to be separated during normal use.

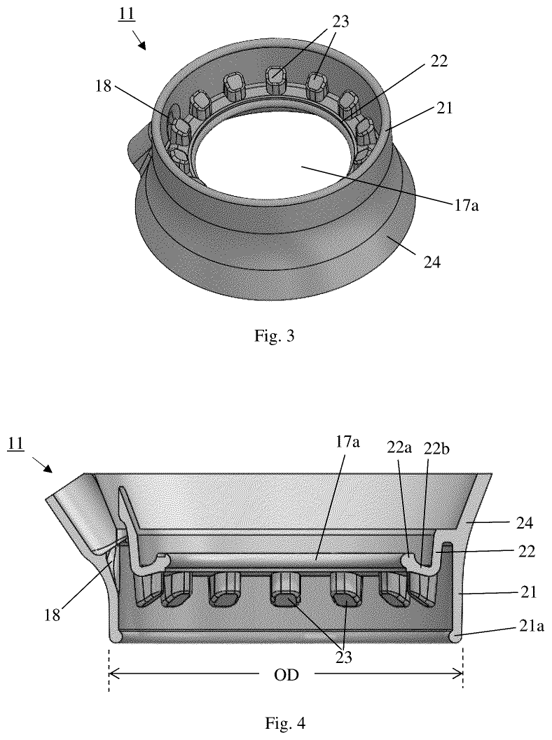

FIG. 3 is a bottom perspective view (oriented upside-down relative to FIG. 1), and FIG. 4 is a side cut-away view, of the suction ring 11 of the patient interface device of FIG. 1. Note that these figures show the suction ring 11 by itself for clarity, but as described earlier, the suction ring 11 is integrally joined to the cone substrate 16. As shown in FIGS. 2, 3 and 4, the suction ring 11 includes a base portion 24 joined to the lower end of the shell 12, and a flexible skirt 21 and a flexible diaphragm 22 extending from the base portion. Both the skirt and the diaphragm are circular in shape. The skirt 21 extends substantially vertically and downwardly from the base portion 24. The diaphragm 22 is disposed inside of the skirt, and recessed with respect to the distal (lower) end 21a of the skirt 21.

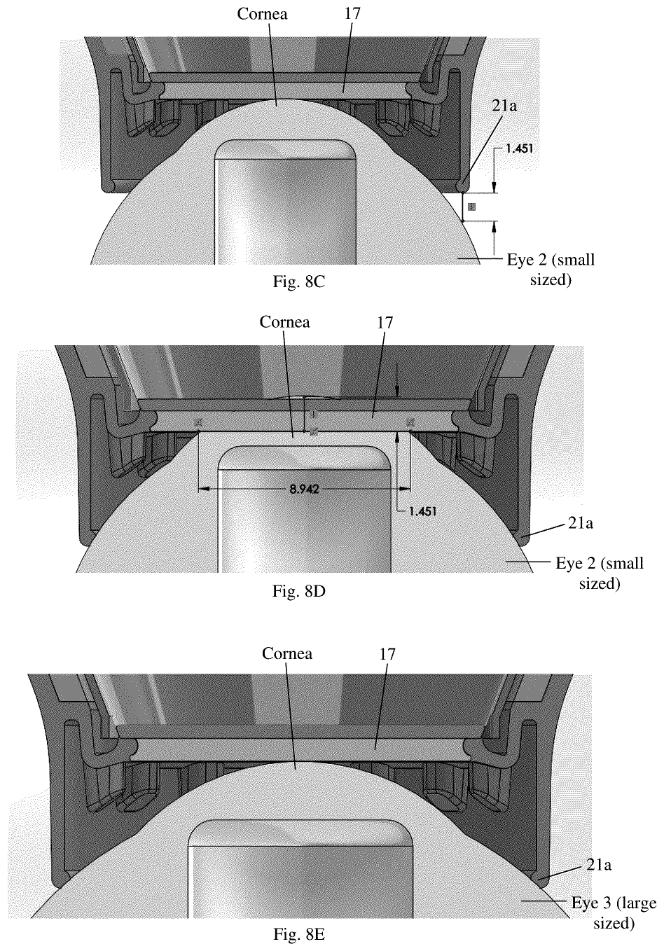

In the illustrated embodiment, the proximal (upper) portion of the diaphragm 22 extends downwardly and is spaced apart from the skirt by a ring shaped gap; the distal portion 22b of the diaphragm 22 bends radially inwardly to form a flange, where the inner circumference of the flange is adapted to hold a contact lens 17 of the patient interface device in the center opening 17a of the diaphragm. More specifically (see FIG. 8A), the contact lens 17, preferably formed of glass, is disc shaped with a groove around its circumferential side surface. The distal end 22a of the diaphragm, i.e., the inner circumference of the flange, fits into the groove to hold the contact lens 17. The cross-sectional shapes of the groove and the distal end 22a of the diaphragm are complementary to each other to securely hold the contact lens. When the patient interface device is coupled to the laser delivery head, the lower end 31 of the laser delivery head (e.g. the objective lens) passes through the shell 12 and contacts the top surface of the contact lens 17 (see FIG. 8A). When the patient interface device is coupled to the patient's eye, the laser delivery head 31 pushes the contact lens 17 against the cornea to applanate the cornea (see FIG. 8B). The lower surface of the contact lens which contacts the cornea may be either flat or curved (e.g. concave). The shape of the diaphragm 22 is not limited to that shown in FIGS. 4 and 8A; other shapes may be used, so long as the diaphragm securely holds the contact lens 17 at a desired position.

In the embodiment shown in FIGS. 2-4, the suction ring 11 further includes a plurality of discrete scleral support pads 23 located inside the skirt 21, joined to and extending downwardly from the bent portion 22b of the diaphragm 22. The scleral support pads 23 are preferably evenly spaced in the circumferential direction. The bottom surface of each scleral support pads 23 is sloped, being lower (closer to the lower rim of the skirt 21) at the radially outer end and higher at the radially inner end. The scleral support pads are optional.

The suction ring 11 of the patient interface further defines a vacuum port 18 which is open on the interior surface of the skirt 21. The other end of the vacuum port is connected to a suction tubing 14 which extends along the tubing guide arm 13 and is ultimately connected to a vacuum source.

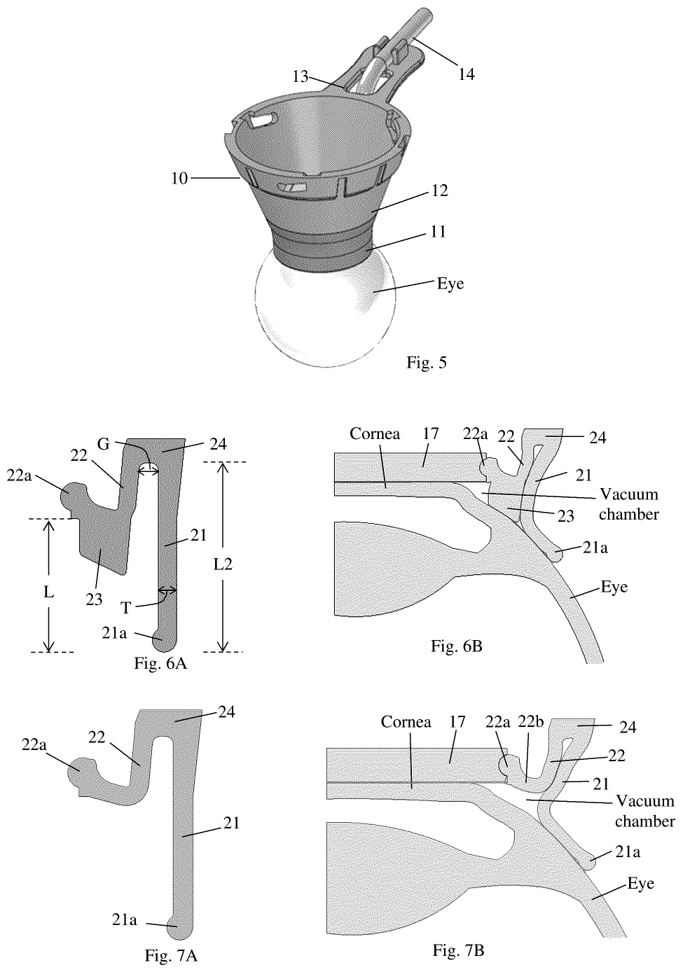

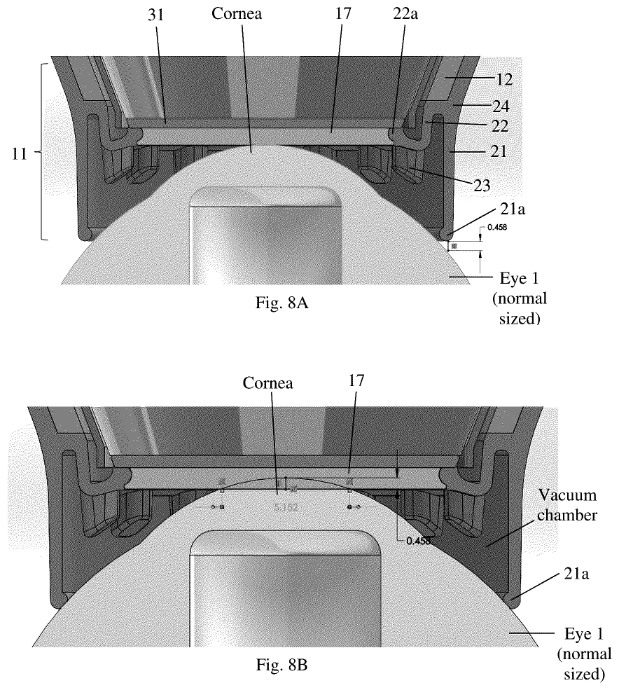

When the patient interface device is placed on the patient's eye and the skirt 21 contacts the surface of the eye, the skirt, the diaphragm, the contact lens 17 and the surface of the eye form a vacuum chamber (see FIG. 8B), which is sealed except for the vacuum port 18. By applying a vacuum pressure to the vacuum chamber via the vacuum port 18 and the suction tubing 14, the suction ring 11 can be securely coupled to the patient's eye (see FIG. 5). FIG. 6B (cross-sectional view of a part of the suction ring, the contact lens, and the eye) illustrates the deformed shape of the suction ring under the vacuum pressure, as compared to the undeformed suction ring shown in FIG. 6A. It can be seen that when the contact lens applanates the cornea and the vacuum pressure is applied, the skirt 21 curves inwardly under the vacuum force, and a substantial portion of the lower part of the skirt contacts and extends along the eye surface, forming a tight vacuum seal. The lower end of the scleral support pads 23 also contact the eye surface when the vacuum pressure is applied. FIGS. 7A and 7B are similar views as FIGS. 6A and 6B, but are for a suction ring that does not have any scleral support pads. Note that in either case, the diaphragm 22 (excluding the scleral support pads) does not contact the eye surface when the vacuum pressure is applied, but the upper portion of the diaphragm 22 may be deformed as it is pushed inwardly by the skirt 21.

During the ophthalmic procedure prior to laser treatment, to couple the eye to the laser delivery head, the patient interface device 10 is first installed on the laser delivery head, e.g. by engaging a plurality of bayonet pin slots 15 (see FIG. 1) on the shell 12 with corresponding protruding pins on the laser delivery head (not shown in the drawings). The patient and the laser delivery head are brought closer to each other (e.g., by moving the laser delivery head and/or the patient bed), until the suction ring 11 of the patient interface device, in particular, the distal end of the skirt 21, contacts the patient's eye. A vacuum is then applied to the suction ring via the suction tubing 14 to affix the patient interface to the eye.

A challenge in patient interface design is to ensure that the device is suitable for use with eyes of different sizes. A typical or “normal sized” human eye is approximately 24 mm in diameter, but smaller eyes may be as small as 21 mm in diameter and larger eyes may be as large as 27 mm in diameter. The same patient interface device, when used with different eye sizes, will fit differently as illustrated in FIGS. 8A-8E.

FIGS. 8A-8E schematically illustrate the patient interface device, coupled to the laser delivery head such that the lower end of the laser delivery head 31 pushes against the contact lens 17, in various spatial relationships relative to various eye models. No vacuum suction is applied. FIG. 8A shows the suction ring placed on a model of a normal sized eye (24 mm diameter). Without applanating the cornea (i.e., the contact lens 17 just touches the top of the cornea but does not applanate it), the lower end 21a of the skirt 21 cannot reach the eye surface. For the illustrated suction ring model, the gap between the lower end 21a of the skirt and the eye surface is 0.458 mm. FIG. 8B shows the same suction ring with the same eye model, but the cornea is applanated by the contact lens 17 by a small amount (0.458 mm vertical travel in this example, resulting in a applanated cornea area of approximately 5 mm in diameter, referred to as the meniscus) before the lower end 21a can touch the eye surface to form a seal.

FIG. 8C shows the same suction ring placed on a model of a smaller sized eye (21 mm diameter). Without applanating the cornea, the lower end 21a of the skirt 21 cannot reach the eye surface, and a gap (1.451 mm in the illustrated example) exists between the lower end 21a and the eye surface. The gap is larger than in the case of the normal sized eye. Thus, as shown in FIG. 8D, the cornea needs to be applanated more for the smaller eye model, in this example by 1.451 mm in vertical travel (resulting in approximately 9 mm diameter of applanated cornea area) before the lower end 21a can touch the eye surface to form a seal.

FIG. 8E shows the same suction ring placed on a model of a larger sized eye (27 mm diameter). For this eye size, the lens to cornea contact and the skirt to eye contact occur approximately simultaneously, i.e., no applanation is required for the skirt to touch the eye surface to form a seal.

In preferred embodiments of the present invention, various parameters of the suction ring 11 are optimized to accommodate a wider range of eye sizes and shapes, in particular, smaller eyes, while achieving desired performance objectives. The objectives include reducing interference with orbital bones of the patient, reducing contact pressure on the eye, reducing intraocular pressure (IOP) increase, preventing eye rolling movements, etc. The preferred and optimum ranges of the various parameters are chosen based on numerical modeling, bench studies, and clinical tests. The analyses and studies conducted include finite element analysis (FEA) simulations of various models, video observations of the docking process, IOP measurements, etc. During bench top studies, a patient interface device with the optimized parameters was able to fit different sized eyes and minimize eye shifts. Data analysis confirmed pupil movement to be less than 300 microns on several trials using such patient interface devices.

With reference to FIG. 4 and FIG. 6A, the parameters of the suction ring and their preferred values are described below. Note that the definitions of parameters for the suction ring in FIG. 7A which has no scleral support pads are the same.

(1) Skirt outer diameter (OD): The outer diameter of the skirt 21 at the lower end. The skirt OD is preferably 14 mm-21 mm, and more preferably, 15-19 mm. In one particular example, the skirt OD is 19 mm. Numerical modeling showed that, within the preferred range, increasing the skirt diameter increases IOP but decreases contact force at the side of the skirt. The lower limit of the OD is constrained by the size of the contact lens, which is preferably approximately 13 mm. The inventors have tested outer diameter values between 15 mm and 20 mm and determined their operability, and believe that OD between 14 mm and 21 mm is generally suitable.

(2) Skirt length (L): The vertical (axial) distance from the bottom of the contact lens 17 to the lower end 21a of the skirt 21. The skirt length is preferably 3 mm-6.5 mm, and more preferably, 5.47 mm-5.52 mm. In one particular example, the skirt length is 5.5 mm. Numerical modeling showed that longer skirt lengths better accommodates optimal applanation level at the time when vacuum is engaged (see discussion of FIGS. 8A-8E), and increases rolling resistance, but also increases IOP. Lab testing showed that longer skirts reduced applanation force and meniscus at the capture point (point of seal formation). In some lab tests, a patient interface device with a warped skirt length was compared to a patient interface device with non warped skirt; it was demonstrated that the patient interface device with no warpage exhibited less eye shift or movement when the vacuum was applied, than the patient interface device with a warped skirt. Additional tests showed that with smaller skirt OD, e.g. 16 mm, skirt lengths as low as 3.1 mm successfully achieved vacuum seal.

As can be understood from the earlier discussion regarding FIGS. 8A-8E, the skirt length L defined from the bottom of the contact lens, as opposed to the total length L2 of the skirt from the base (where the skirt 21 and diaphragm 22 both join the base portion 24, see FIG. 6A), impacts the amount of applanation required before the lower end of the skirt contacts the eye surface. The skirt length L as defined above is physically determined by both the length of the skirt and the length of the diaphragm. Thus, both the skirt and the diaphragm may be lengthened or shortened while maintaining the skirt length L unchanged. The total length of the skirt L2 itself is less important. In the above example where L=5.5 mm, the total length L2 of the skirt is L2=7.1 mm, but this value is not limiting, and other values may be used.

(3) Skirt thickness (T): The thickness of the skirt. The skirt thickness is preferably 0.25 mm-3 mm, and more preferably, 0.42 to 0.62 mm. In one particular example, the skirt thickness is 0.5 mm. Within the preferred range, the skirt thickness has a relatively small impact on contact force and IOP and the contact force at the side of the skirt, but higher thickness correlated with higher contact force at the end of the skirt. Thickness greater than the preferred range may cause the skirt to be too stiff.

(4) Skirt gap width (G): The width of the gap between the skirt and the diaphragm at the location where both join the base portion 24. The gap width is preferably 0.55 mm, but values between 0.1 and 0.75 may be used. This parameter does not significantly impact the suction ring performance.

(5) Stiffness of the skirt material: Preferably 20A-70A durometer (Shore hardness), and more preferably, 30A-42A. Numerical modeling showed that within the preferred range, increased stiffness caused a decrease in IOP and a decrease in contact force at the skirt, with or without scleral support pads. It also showed that a stiffer skirt, in conjunction with the scleral support pads, shifted the load to the edge of the skirt, which is desirable in grasping the loose conjunctiva tissue. In some tests, when the material was too stiff (e.g. 95A), it did not conform to the eye and could not hold a vacuum.

(6) Scleral support pads: As mentioned earlier, the scleral support pads are optional. Note that FIGS. 6A and 6B correspond to a suction ring with scleral support pads 23, while FIGS. 7A and 7B correspond to a suction ring without scleral support pads. When present, the scleral support pads contact the sclera of the eye when the patient interface is coupled to the eye. They help to maintain the structural integrity of the sclera when the vacuum is initiated, to reduce IOP increase, while still achieving a good vacuum seal between the suction ring and the eye. The scleral support pads function to prevent major deformation of the sclera when the cornea is applanated.

In embodiments where scleral support pads are used, the width and surface area of the pads are optimized to achieve the above objectives. In one particular embodiment, 14 scleral support pads are provided and even spaced from each other. Their width in the circumferential direction is approximately 1.63 mm, and their width in the radial direction is approximately 1.56 mm. Their vertical length, measured from the bottom of the contact lens, is approximately 1.20 mm at the radially inner end and 1.91 mm at the radially outer end. These dimensions are not limiting, and other values may be used.

(7) Skirt flare: In the embodiments shown in FIGS. 1-8E, the skirt 21 extends substantially vertically from the base 24. In some alternative embodiments, the skirt may have a slight flare, which may help with the compression of the suction ring and fit with the eye. A flare within the range of 5-8 degrees is preferred. Tests showed that a flare of 10 degrees did not fit well with the eye when the skirt lengths were from 5.3 mm to 6.6 mm.

The above listed parameters were adjusted independently in the optimization analyses and testing. However, numerical modeling and bench tests showed that the effects of changing one parameter often depend on the values of other parameters as well as the eye size. The above preferred parameter value ranges were obtained after extensive analyses and testing, and were determined to be combinations that optimize the overall performance considering multiple objectives.

Tests were also conducted to compare the single-piece patient interface device with a two-piece patient interface device. The single-piece patient interface device reduced docking procedure time by at least 30 seconds. This will in turn reduce patient time under vacuum. Tests also showed over 50% improved eye rolling resistance when compared to the two-piece device. IOP rise was measured approximately 15%-20% less compared to IOP rise with the two-piece comparison device. Tests also confirmed easier meniscus formation beyond 10 mm diameter, creation of a fewer number of corneal folds, ease of use, and reduced workflow steps. Additionally, the tubing length was reduced for a convenient length to use during the procedures with a lower profile. In addition, single-piece patient interface devices eliminate any chances of relative mechanical motions between the pieces, and reduce the number of parts needed for surgery.

The patient interface device described herein may be used for both the left and right eyes, by rotating the device (e.g., by about 120 degrees, or other suitable angles) such that the tubing guide arm 13 and the suction tubing 14 extend toward the temple of the patient.

In some alternative embodiments, two or more vacuum ports are provided in the suction ring, which may reduce the chance of eye movement during vacuum application.

To summarizes, single-piece patient interface devices according to embodiments of the present invention have the following advantages: reduced skirt outer diameter; accommodating a wider range of eyes to fit; reduced interference with orbital bones that can cause difficult docking; reduced occurrence and size of corneal folds during clinical trials; etc.

The single-piece patient interface device according to embodiments of the present invention is suitable for use in corneal flap and corneal lenticule procedures. Moreover, the patient interface device may be used for refractive index modification of the crystalline lens, cataract surgeries, or other laser applications that requires holding the patient eye, keeping the eye stationary while connecting the eye to the laser systems.

It will be apparent to those skilled in the art that various modification and variations can be made in the patient interface device of the present invention without departing from the spirit or scope of the invention. Thus, it is intended that the present invention cover modifications and variations that come within the scope of the appended claims and their equivalents.

1. A patient interface device for coupling a patient's eye to an ophthalmic surgical laser system, comprising:
a cone substrate, including a rigid frustoconical shaped shell, configured to be coupled to the ophthalmic surgical laser system; and
a flexible suction ring joined to a lower end of the rigid shell,
wherein the cone substrate and the suction ring are formed integrally as a single piece,
wherein the suction ring includes a base portion joined to the lower end of the rigid shell, a circular skirt extending downwardly from the base portion, a circular diaphragm extending from the base portion and disposed inside of the skirt, and a contact lens joined to the diaphragm and disposed in a center opening of the diaphragm, and wherein when the skirt contacts a surface of the patient's eye, the skirt, the diaphragm, the contact lens and the surface of the eye form a vacuum chamber.

2. The patient interface device of claim 1, wherein an outer diameter of the skirt is between 14 mm and 21 mm, a skirt length defined as a vertical distance from a lower surface of the contact lens to a distal end of the skirt is between 3 mm and 6.5 mm, a thickness of the skirt is between 0.25 mm and 3 mm, and a stiffness of the skirt is between 20A and 70A Shore durometer.

3. The patient interface device of claim 2, wherein the outer diameter of the skirt is between 15 mm and 19 mm, the skirt length is between 5.47 mm and 5.52 mm, the thickness of the skirt is between 0.42 and 0.62 mm, and the stiffness of the skirt is between 30A and 42A Shore durometer.

4. The patient interface device of claim 2, wherein the outer diameter of the skirt is 19 mm, the skirt length is 5.5 mm, and the thickness of the skirt is 0.5 mm.

5. The patient interface device of claim 2, wherein a width of a gap between the skirt and the diaphragm at locations where both join the base portion is approximately 5.5 mm.

6. The patient interface device of claim 1, wherein the skirt extends from the base in a vertical direction.

7. The patient interface device of claim 1, wherein the skirt extends from the base with a flare of 5 to 8 degrees.

8. The patient interface device of claim 1, wherein a distal portion of the diaphragm extends radially inwardly to form a flange, and wherein the contact lens is joined to an inner circumference of the flange.

9. The patient interface device of claim 1, wherein the suction ring further includes a plurality of scleral support pads joined to the diaphragm and extending downwardly and evenly spaced in a circumferential direction, and wherein a bottom surface of each scleral support pad is sloped, being lower at a radially outer end and higher at a radially inner end.

10. The patient interface device of claim 9, wherein a width of the scleral support pads in the circumferential direction is approximately 1.63 mm, a width of the scleral support pads in the radial direction is approximately 1.56 mm, a vertical length of the scleral support pads defined from a bottom of the contact lens is approximately 1.20 mm at a radially inner end and 1.91 mm at a radially outer end.

11. The patient interface device of claim 1, wherein the suction ring defines at least one vacuum port open on an interior surface of the skirt, the patient interface device further including a suction tubing coupled to another end of the vacuum port.

12. The patient interface device of claim 11, wherein the cone substrate further includes a tubing guide arm that extends from the shell, and wherein the suction tubing extends along the tubing guide arm.

13. The patient interface device of claim 1, wherein cone substrate is formed of a hard plastic material.

14. The patient interface device of claim 1, wherein the suction ring is formed of a soft thermoplastic elastomer material.

15. The patient interface device of claim 1, wherein the cone substrate and the suction ring are formed integrally by overmolding.

16. The patient interface device of claim 1, wherein the cone substrate includes a plurality of slot features configured to attach the patient interface device to the ophthalmic surgical laser system.

17. The patient interface device of claim 1, wherein a lower surface of the contact lens is either flat or curved.
